(19) United States
(12) Patent Application Publication
(10) Pub. No.: US 2026/0007841 A1
(43) Pub. Date: Jan. 8, 2026

(54) ERGONOMIC AND UTILITARIAN AEROSOL DELIVERY SYSTEM

(71) Applicant: KAER BIOTHERAPEUTICS CORPORATION, Escondido, CA (US)

(72) Inventors: Donovan B. Yeates, Escondido, CA (US); Sai Siva Kare, Escondido, CA (US); Milad Samaee, Aliso Viejo, CA (US)

(21) Appl. No.: 19/257,463

(22) Filed: Jul. 2, 2025

Related U.S. Application Data
(60) Provisional application No. 63/667,670, filed on Jul. 3, 2024.

(51) Int. Cl. A61M 15/00; A61M 39/22

(52) U.S. Cl. CPC A61M 15/009; A61M 15/002; A61M 39/227; A61M 2205/0238; A61M 2205/3344; A61M 2209/084; A61M 2240/00

(57) ABSTRACT

An aqueous aerosol generation and delivery system for high dose aerosol delivery, having a multistage pod having an inner cavity comprising in series a plurality of sequentially arranged multi-stage conical cavities having the shape of truncated cones narrowing in a direction of flow of the aerosol. The rounded annulus cavity directs dilution gas towards the nozzle tip. The plurality of sequentially arranged multi-stage conical cavities are disposed between the nozzle tip and the output end within the multistage pod. The multi-stage conical cavities are configured to decrease the velocity of the aerosol plume by having said shape of truncated cones narrowing in a direction of flow of the aerosol, such that the system expels a dense column of soft-flowing aerosol suitable for inhalation by a patient, as well as for animal respiratory healthcare.

### CROSS-REFERENCE TO RELATED APPLICATION

This application claims the priority of the U.S. Provisional Application 63/667,670, having a filing date of Jul. 3, 2024. The entire content of this prior application 63/667,670 is herewith incorporated by reference.

### BACKGROUND OF THE INVENTION

AeroPulsR is a unique device developed to overcome inadequate delivery of low and high molecular weight and structure over a range on viscosities from 1 cP to at least 50 cP therapeutic aerosols to pediatric and adult patients to resolve the underlying etiologies and consequent respiratory distress. The United States patent application publications US20240399099A1 and US20240399079A1 as well as the international patent application publications WO 2024/252288 A1 and WO 2024/252306 A1 provide the descriptions of the underlying device and system. The functional utility of this unique device, AeroPulsR, has been markedly improved by inclusive of interaction operation by human use as well as generate and deliver the inhalation of the broad spectrum of therapeutics by pediatric and adults, as well as for animal respiratory healthcare.

### SUMMARY OF THE INVENTION

According to a first aspect of the invention, an aqueous aerosol generation and delivery system for high dose aerosol delivery is provided, said system comprising: a multistage pod having an inner cavity comprising in series a plurality of sequentially arranged multi-stage conical cavities having the shape of truncated cones narrowing in a direction of flow of the aerosol, said multistage pod further comprising: an input end comprising an aerosolizing nozzle having a fluid input port and being inserted into a cylindrical channel and, a nozzle gas port, and a dilution gas port configured to receive an aerosolizing nozzle; and an output end comprising an output opening through which aerosol is delivered; wherein the aerosolizing nozzle is configured to generate and expel aerosol through a nozzle tip; a rounded annulus cavity disposed within the multistage pod proximate to the nozzle tip, said rounded annulus cavity configured to direct dilution gas towards the nozzle tip; and said plurality of sequentially arranged multi-stage conical cavities are disposed between the nozzle tip and the output end within the multistage pod, wherein the multi-stage conical cavities are arranged coaxial with the nozzle, and wherein the multi-stage conical cavities are configured to decrease the velocity of the aerosol plume by having said shape of truncated cones narrowing in a direction of flow of the aerosol, such that the system expels a dense column of soft-flowing aerosol suitable for inhalation by a patient; wherein the input end of the multistage pod further includes: a nozzle gas port configured to supply high-pressure gas to a nozzle annulus via a T-channel, said nozzle annulus circumferentially surrounding the plurality of nozzle gas input holes; and a dilution gas port configured to supply low-pressure gas to a rounded annulus cavity via an L-channel.

According to a second aspect of the invention, an aqueous aerosol generation and delivery system is provided, said system comprising: a chamber having a chamber input end and a chamber output end; an aerosol generating nozzle configured to be inserted into a nozzle input port formed in the chamber input end, said nozzle comprising a nozzle barrel having a plurality of circumferentially arranged nozzle gas input holes; a cone having a first cone end and a second cone end, wherein: the first cone end is configured to attached to the chamber output end; the diameter of the first cone end is greater than the diameter of the second cone end; and the second cone end includes an opening configured to expel aerosol generated by the nozzle; and a counterflow tube arranged within the chamber, said counterflow tube configured to eject pressured gas coaxially in the opposite direction of the nozzle to decelerate the high velocity aerosol emanating from the nozzle; wherein the chamber input end further includes: a nozzle gas port configured to supply high-pressure gas to a nozzle annulus via a T-channel, said nozzle annulus circumferentially surrounding the plurality of nozzle gas input holes; and a counterflow gas port configured to supply low-pressure gas to the counterflow tube via a straight channel; wherein the nozzle gas port and the counterflow gas port are separate ports that are not interconnected; the counterflow gas port is the only source of dilution gas; and the system is configured to expel a dense column of soft-flowing aerosol suitable for inhalation by a patient.

### DETAILED DESCRIPTION OF THE INVENTION

Critical respiratory insufficiency such as acute respiratory distress syndrome (ARDS) and acute lung injury (ALI) are exacerbated by abnormal or reduced lung surfactant. Surfactant lining the alveoli enables expansion of the lung and facilitates oxygen and carbon dioxide exchange. A proposed treatment is the administration of surfactant aerosols delivered to the deep lung. AeroPulsR is a device that is designed to aerosolize surfactant (and other agents) and deliver the aerosol to the deep lung. These other agents may include antibiotics and proteins amongst other therapeutic drugs. A schematic of AeroPulsR is shown in FIG. 1.

The present invention contemplates and solves a variety of objective technical problems through the specific structural features and method steps disclosed in this description.

A first objective of the aerosol generation and delivery system is to improve and simplify the functionality of high concentration aerosol drug dose delivery and end user's (e.g., a patient or clinician) interaction with the aerosol generation and delivery system.

A second objective of the aerosol generation and delivery system is to accommodate the main aerosol generating nozzle in a pod shaped chamber such that the nozzle is easily inserted for therapy, and easily removed from the chamber for maintenance and sterilization purposes.

A third objective of the aerosol generation and delivery system is to accommodate an optimized gas flow through a counter-flow tube in the pod shaped chamber decelerates the high velocity aerosol emanating from the nozzle. This counter-flow gas is used to optimize the aerosol concentration and may include aerosol generation using heliox and dilution using an economical gas such as compressed air or compressed oxygen.

A fourth objective of the aerosol generation and delivery system is to accommodate independent pneumatic circuits for the nozzle and the counter-flow tube such that the additional use of pneumatic tubing from chamber to nozzle and from chamber to counterflow tube are omitted.

A fifth objective of the aerosol generation and delivery system is to adapt an aerosol delivery cone with the chamber through a lip seal. The cone converges the aerosol emanating from the chamber into a concentrated column of aerosol amenable for inhalation and includes a standard medical taper for adapting further aerosol transport tubing and accessories specific to the aerosol generation and delivery system.

A sixth objective of the aerosol generation and delivery system is to enable simple assembly and disassembly of the chamber, nozzle, counter-flow tube and cone for effective aerosol therapy delivery and sterilization.

A seventh objective of the aerosol generation and delivery system is to accommodate the effect of the cone length on the aerosol output characteristics in terms of aerosol concentration, output efficiency and output latency.

An eighth objective of the aerosol generation and delivery system is to utilize a compact multistage pod design with significantly reduced internal volume when compared with chamber and cone that needs assembly and generate high concentrations of aerosol boluses with short time duration for application in aerosol generation and delivery to pediatric patients.

A ninth objective of the aerosol generation and delivery system is to establish the aerosol output efficiency of selected cone lengths related to the compact multistage pod, for liquid flow aerosol generation rates between 1 and 12 ml/min, such that: (i) a maximum of 32% and a minimum of 20% efficiency at 1 and 12 ml/min is achieved for chamber with 70 mm long cone; (ii) a maximum of 35% and a minimum of 15% efficiency at 1 and 12 ml/min is achieved for chamber with 100 mm long cone; (iii) a maximum of 38% and a minimum of 12% efficiency at 1 and 12 ml/min is achieved for chamber with 150 mm long cone; and (iv) a maximum of 24% and a minimum of 18% efficiency at 1 and 12 ml/min is achieved for the compact multistage pod.

A tenth objective of the aerosol generation and delivery system is to have identical external shape of the aerosol generation and processing system formed of 1) compact multistage pod, and 2) the chamber and cone assembly, therefore, utilizing an identical pedestal and associated mounting bracketing bracket to securely hold the aerosol generation and processing system in place atop the console during therapy or use.

An eleventh objective of the aerosol generation and delivery system is to position the chamber and cone or the compact multistage pod such that it is ergonomically accessible by ambulatory patients.

A twelfth objective of the aerosol generation and delivery system is to position the chamber and cone or the compact multistage pod such that upon securing the chamber to the pedestal and subsequently the pedestal to the rigid mounting bracketing bracket atop the console, the mouthpiece of the cone or the compact multistage pod does not move during use.

A thirteenth objective of the aerosol generation and delivery system is to deliver consistent aerosol boluses of time period of 2 s long at 0.2 Hz suitable for adult patients and 0.5 s long at 0.67 Hz suitable for pediatric patients, over time durations that are stable for at least 10 min without decrease in aerosol volume delivery. It is noted that aerosol generation is also conducted continuously or to periodic bolus delivery. Furthermore, the aerosol generation and delivery system generates aerosol boluses of time duration as short as 30 ms.

A fourteenth objective of the aerosol generation and delivery system is to simplify the interaction between the device and the user, including patient or clinician, such that affective aerosol therapy is achieved with minimum number of inputs or training. This simplification of therapy is achieved primarily achieved by the parameter input panel wherein using a single knob, the patient or clinician inputs the type of aerosol therapy the patient is prescribed: (i) inhalation/exhalation activated aerosol generation and delivery, wherein, once switched on with the liquid drug loaded, upon the initiation of the patient's inhalation the aerosol therapeutic agent is automatically activated and stops during exhalation; and (ii) the patient or clinician inputs the aerosol inhalation and exhalation duration and number of inhalations, and the therapy guidance pilot lights on the console, such that the patient may synchronize his/her breathing with the aerosol generation and delivery timings of the aerosol generation and delivery system.

A fifteenth objective of the aerosol generation and delivery system is to enable the inhalation/exhalation activated aerosol generation and delivery through a modified bidirectional valve which attaches to the aerosol delivery cone, and comprises an inbuilt conduit responsible for detecting pressure differences near the mouth of the patient due to inhalation or exhalation, and relaying this information to a pressure transducer within the console.

### BRIEF DESCRIPTION OF THE DRAWINGS

FIG. 1 shows a top perspective view of an aerosol generation and processing system.

FIG. 2 shows an exploded top side perspective view of components forming the aerosol generation and processing system, including a chamber and cone assembly with pedestal and mounting bracket.

FIG. 3A shows a side view of the chamber and cone assembly connected to a pedestal.

FIG. 3B shows a front view of the chamber and cone assembly connected to a pedestal, including cross-sectional cut line A-A shown in FIG. 3C.

FIG. 3C shows a cross-sectional side view taken along line A-A of FIG. 3B of the chamber and cone assembly connected to the pedestal.

FIG. 4A shows a side view of a chamber, shown independent of other system components.

FIG. 4B shows a front view of the chamber, including cross-sectional cut lines M-M and N-N, the cross-sectional views of which are respectively shown in FIGS. 4C and 4D.

FIG. 4C shows the cross-sectional view of the chamber taken along section M-M of FIG. 4B.

FIG. 4D shows the cross-sectional view of the chamber taken along section N-N of FIG. 4B.

FIG. 5A shows a front view of a mounted chamber and cone assembly, including cross-sectional cut lines B-B and C-C, the cross-sectional views of which are respectively shown in FIGS. 5B and 5C.

FIG. 5B shows a cross-sectional view of the chamber and cone assembly, taken along cut line B-B.

FIG. 5C shows a cross-sectional view of the chamber and cone assembly, taken along cut line C-C.

FIG. 5D shows detail I from cross-section B-B of FIG. 5B, showing the high-pressure gas routing for the nozzle.

FIG. 5E shows detail H from cross-section C-C of FIG. 5C, showing the base of counterflow tube.

FIG. 6A shows a front view of the chamber and cone assembly connected to a pedestal, including cross-sectional cut line H-H shown in FIG. 6B.

FIG. 6B shows a cross-sectional view of the chamber and cone assembly connected to a pedestal taken along cut line H-H, and including detail views J, K, and L.

FIG. 7 shows a top perspective view of the pedestal and mounting bracket in a detached configuration.

FIG. 8A shows a side view of a compact multistage pod assembly attached to a pedestal and mounting plate.

FIG. 8B shows a front view of a compact multistage pod assembly attached to a pedestal and mounting plate, including cross-sectional cut line G-G shown in FIG. 8C.

FIG. 8C shows a cross-sectional side view of a compact multistage pod assembly attached to a pedestal and mounting plate taken along line G-G.

FIG. 9A shows a side view of a compact multistage pod, shown independent of other system components.

FIG. 9B shows a bottom view of the compact multistage pod, including cross-sectional cut lines E-E and F-F, the cross-sectional views of which are respectively shown in FIGS. 9C and 9D.

FIG. 9C shows the cross-sectional view of the compact multistage pod taken along section E-E.

FIG. 9D shows the cross-sectional view of the compact multistage pod taken along section F-F.

FIG. 10A shows a schematic cross-sectional diagram of an exemplary 70 mm length cone.

FIG. 10B shows a schematic cross-sectional diagram of an exemplary 100 mm length cone.

FIG. 10C shows a schematic cross-sectional diagram of an exemplary 150 mm length cone.

FIG. 11 shows a plot of flow rate versus efficiency for a compact multistage pod and conventional chamber and cone assemblies having varying length cones.

FIG. 12A shows a still image of a compact multistage pod outputting aerosol from a mouthpiece.

FIG. 12B shows a still image of a 70 mm length conventional cone outputting aerosol from a mouthpiece.

FIG. 12C shows a still image of a 100 mm length conventional cone outputting aerosol from a mouthpiece.

FIG. 12D shows a still image of a 150 mm length conventional cone outputting aerosol from a mouthpiece.

FIG. 13A shows a still image of a compact multistage pod outputting aerosol from a mouthpiece.

FIG. 13B shows a still image of a 70 mm length conventional cone outputting aerosol from a mouthpiece.

FIG. 13C shows a still image of a 100 mm length conventional cone outputting aerosol from a mouthpiece.

FIG. 13D shows a still image of a 150 mm length conventional cone outputting aerosol from a mouthpiece.

FIG. 14A shows a plot of light scatter intensity as a function of time with for pulsed aerosol generation with an on-duration of 2 seconds and an off-duration of 3 seconds, along with inlaid images of aerosol output at various time increments.

FIG. 14B shows a plot of light scatter intensity as a function of time with for pulsed aerosol generation with an on-duration of 0.5 seconds and an off-duration of 1 second, along with inlaid images of aerosol output at various time increments.

FIG. 15A shows a schematic top perspective view of the modified bidirectional valve assembly attached to an output cone.

FIG. 15B shows a schematic top perspective view of the modified bidirectional valve assembly shown in isolation.

FIG. 15C shows a rear view of the modified bidirectional valve assembly, including cross-sectional cut lines I-I and J-J, the cross-sectional views of which are respectively shown in FIGS. 15D and 15E.

FIG. 15D shows the cross-sectional view of the modified bidirectional valve assembly taken along section I-I.

FIG. 15E shows the cross-sectional view of the modified bidirectional valve assembly taken along section J-J.

FIG. 16A shows a cross-sectional view of the modified bidirectional valve with the flap in a closed position.

FIG. 16B shows a cross-sectional view of the modified bidirectional valve with the flap in an open position.

### DETAILED DESCRIPTION OF THE DRAWINGS

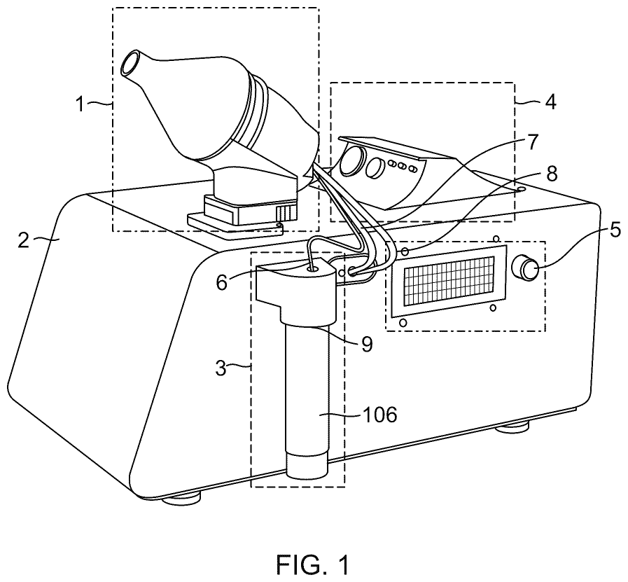

FIG. 1 shows the complete AeroPulsR therapeutic device. The aerosol generation and processing system 1 is attached to the top of the console 2 by a mounting bracket. The console 2 may be located, for instance, beside a chair or bed for patient ease of therapeutic aerosol use. The drug is loaded in the dispenser system 3. According to an embodiment, the drug is a liquid that is loaded in a vial 106 that is inserted using a ½ turn into a dispenser system 3 that has a matching single handed ½ turn. The console 2 may include a parameter input panel 5, which may include a rotary dial and an LCD display. The treatment protocol may be loaded and treatment parameters are set on a digital LCD display using a rotary dial. For instance, the tidal volume, frequency and treatment period may be selected using a single rotary dial displayed on the parameter input panel 5. The console 2 may further include a device control panel 4, which may include a button and guiding lights. When ready, a patient or clinician may initiate the treatment by pressing a green button on the device control panel 4 atop the console 2. The pressing of the button thus initiates the aerosol treatment, and onboard lights guide the patient to breath. Within the aerosol generation and processing system 1, pneumatic lines 7, 8 supply compressed air for aerosolization and compressed air for diluting and arresting the aerosol by a counterflow tube 17, respectively, whereas a drug is transported from the dispenser system 3 using a flexible capillary 6. Both the pressures and the type of gas fed through the respective pneumatic lines 7 and 8 may be different. For example, heliox has proven advantageous as a nozzle gas for aerosolization since the properties of heliox such as its Reynolds number are advantageous for generating the aerosol, while for arresting the aerosol plume and for dilution cheaper gases such as air may be sufficient. Further, for aerosolization, high pressure may be advantageous, while for dilution a lower pressure may be sufficient.

The aerosol generation and processing system 1 atop the console 2 is designed for modularity, and the components are readily assembled or disassembled for cleaning. The utility aerosol generation, processing and delivery system 1 described in this invention are designed for robustness, and easy use by the patient or operator, as well as facilitate effective cleaning of the components before and after use.

A dispenser system 3 attaches to the console 2 and feeds a liquid drug in the vial 106 through a flexible capillary tube 6 to the nozzle 13, where it is aerosolized as a plume within the chamber 10. A one-way check valve 9 may be inserted between the capillary 6 within the dispenser system 3, prior to the nozzle 13, to enable liquid transport from the dispenser system 3. This one-way check valve 9 prevents reverse liquid transport, thus ensuring fluid availability within the capillary 6 and aerosolizing nozzle 13 for rapid aerosol delivery.

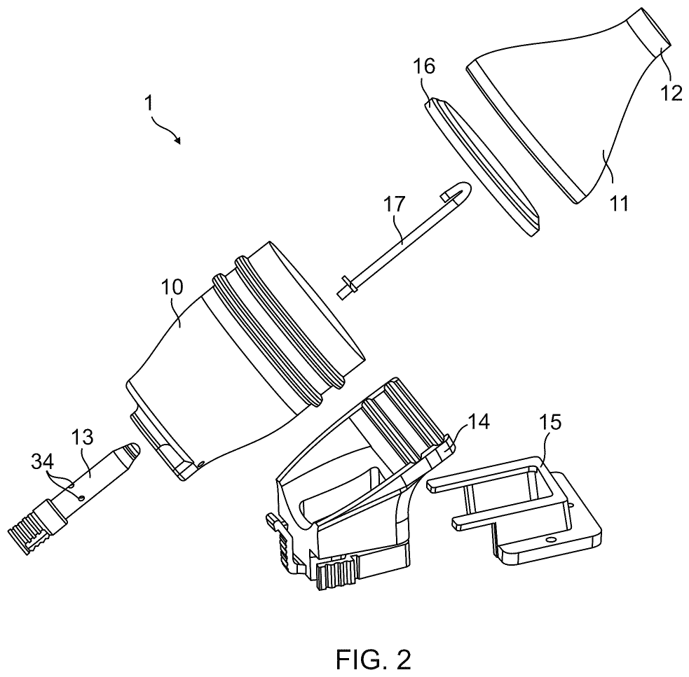

FIG. 2 shows an exploded top side perspective view of the components forming the aerosol generation and processing system 1, pedestal 14, and mounting bracket 15. According to the invention, aerosol is generated and delivered by the aerosol generation and processing system 1. The aerosol generation and processing system 1 comprises a nozzle 13, an aerosol chamber 10, a cone 11 and a counterflow tube 17. In this assembly, a nozzle 13 is inserted into a cylindrical hole located at a first end of the chamber 10, specifically its rear. The nozzle 13 may include gas input ports 34 formed into the body of the nozzle 13, which may reduce gas flow resistance to facilitate rapid activation and arresting of aerosol generation. A counterflow tube 17 is housed within the chamber 10 and is configured to decelerate a fast ejected aerosol plume that is ejected into the chamber 10 by the nozzle 13. A cone 11 is fitted on the chamber 10 using a lip seal 16 disposed between a second end of chamber 10 and a first end of cone 11. The lip seal 16 enables easy attachment and removal of cone 11 from the aerosol chamber 10 for cleaning purposes. According to an alternative embodiment, the lip seal 16 could be replaced using tapered mating surfaces on the chamber 10 and the cone 11. The cone 11 includes a mouthpiece 12 at its second end, by which the aerosol is delivered to the user. The mouthpiece 12 may include a standard medical-type tapered design for adapting aerosol delivery tubing or accessories. The chamber and cone assembly are secured on the pedestal 14 which is then slid into the mounting bracket 15 fastened to the device console 2.

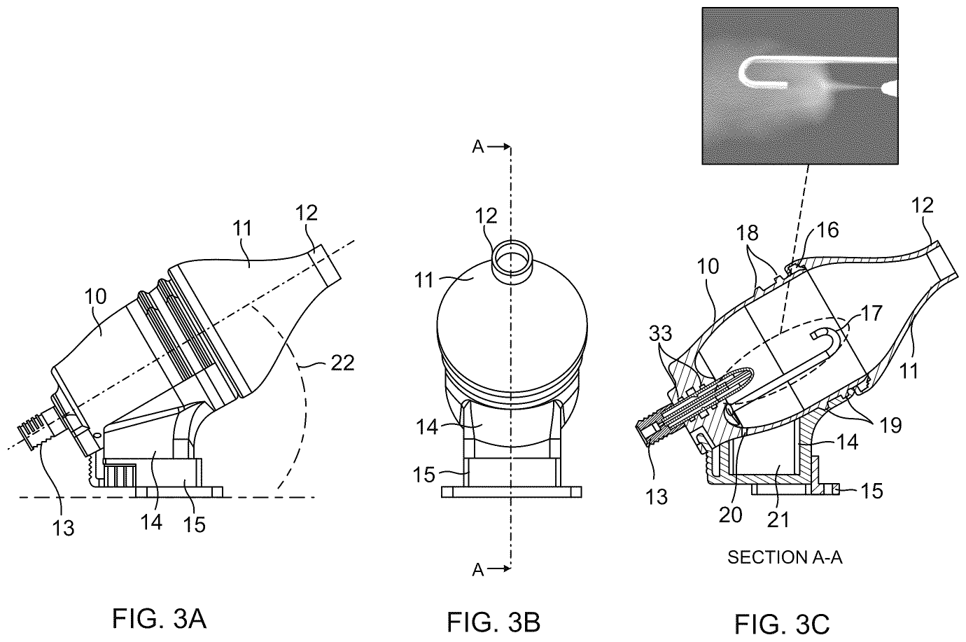

FIGS. 3A-C show various views of a fully assembled chamber and cone assembly connected with a pedestal 14. FIG. 3A shows a side view of the assembled system comprising the nozzle 13, chamber 10, counterflow tube 17 (not visible), cone 11, pedestal 14 and mounting bracket 15. According to a preferred embodiment, the chamber and cone assembly is ergonomically positioned at a 30° angle 22 This angle was found to be comfortable for patients who were situated beside the AeroPulsR to readily place their mouth over a mouthpiece 12 attached at the end of the cone 11.

FIG. 3B shows a front view of the chamber and cone assembly, with a cross-section line A-A included at its central axis. The front view shows the chamber and cone assembly placed on top of pedestal 14, which is on top of mounting bracket 15. The cone 11 is shown with a circular mouthpiece 12 protruding outward at an upwards angle.

FIG. 3C shows a cross-sectional side view of the internal components of the fully assembled chamber and cone assembly. A reservoir 21 is included within a cavity formed within the pedestal 14. Pedestal 14 interfaces with the chamber 10. The chamber and cone assembly is positioned on a pedestal 14 with slots 19 which mate with corresponding ribs 18 on the chamber 10. The reservoir 21 in the pedestal 14 collects the aerosol deposition forming in the cone 11 and chamber 10 through a drain port 20 positioned at the lowest point of the chamber 10. One end of chamber 10 is connected to a cone 11, securely fitted thereto with an intermediate lip seal 16. The lip seal 16 enables leak proof fit between the cone 11 and the chamber 10. The structure of cone 11 converges in the direction moving away from chamber 10. A mouthpiece 12 is included at the end of cone 11 opposite of the end connected with chamber 10. The rear of chamber 10 may include an input port along aligned with the central axis of chamber 10, for instance a cylindrical hole, into which a nozzle 13 may be inserted. The nozzle 13 may be secured within the chamber 10 by two seated O-rings 33. The nozzle 13 is configured to expel a high velocity aerosol plume. Within the chamber 10, the generated aerosol plume is radially redirected by a pressurized gas jet flowing coaxially in the opposite direction through the counterflow tube 17. The counterflow tube 17 disposed within the chamber 10 may be defined by a J-shaped structure, and the base of counterflow tube 17 may be offset from the central axis of the chamber and cone assembly, namely the axis along which nozzle 13 is oriented. The opposite end of the counterflow tube 17 loops such that it is in alignment with and faces the nozzle 13. The counterflow tube 17 is configured to expel gas in a direction opposite to that in which the nozzle 13 expels an aerosol plume. As a result of this configuration, the gas from the counterflow tube 17 baffles the high velocity aerosol plume from the nozzle 13. This baffling function is shown in the insert showing the radially redirected aerosol resulting from the plume colliding with the counterflow tube 17. The decelerated aerosol plume converges through cone 11 to the tapered output mouthpiece 12. This tapered output 12 is designed to accept additional components (e.g., in-line aerosol concentrators or flexible delivery tube for remote aerosol delivery) to facilitate the delivery of the aerosol. The taper of mouthpiece 12 can be modified or eliminated to accept specific aerosol delivery attachments as needed. According to an embodiment, several tapered cones 11 may be included to allow for oral inhalation of the aerosol, or provisions for adapting aerosol delivery tubes.

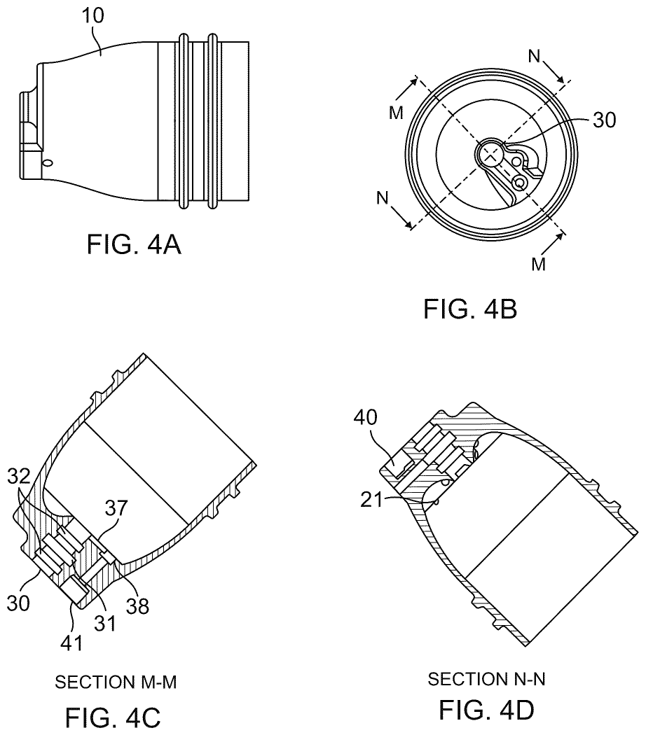

FIGS. 4A-D show various views of the design of the chamber 10. FIG. 4A shows a side view of chamber 10, shown independent of other system components.

FIG. 4B shows a front view of the chamber 10, including cross-sectional cut lines M-M and N-N, the cross-sectional views of which are respectively shown in FIGS. 4C and 4D.

FIG. 4C shows the cross-sectional view of chamber 10 taken along section M-M of FIG. 4B, while FIG. 4D shows the cross-sectional view of chamber 10 taken along section N-N of FIG. 4B. In the following, the components of the chamber 10 are jointly discussed in connection with FIGS. 4C and 4D.

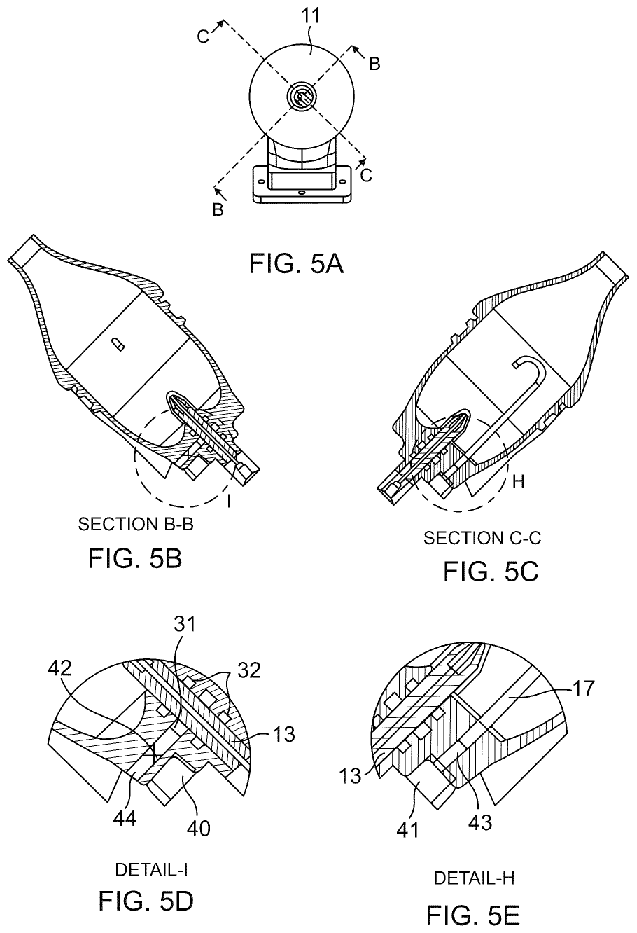

FIGS. 4A-4D illustrate the structure of chamber 10 in isolation, without components that may be inserted into or attached to it. However, for contextual clarity, reference is made below to such components, which are described elsewhere in the specification and/or shown in other figures. The chamber 10 comprises a coaxial cylindrical channel 30 at its rear, configured to receive a nozzle 13 (not shown). This channel 30 includes three annular grooves, namely one major annulus 31 and two minor annuli 32. The central major annulus 31 is larger than the two minor annuli 32 and is configured to deliver compressed gas to the nozzle gas inlet ports 34 (FIG. 2). These two minor grooves 32 are located equidistance on each side of the major annular groove 31. The two minor annuli 32 are coaxial to the central groove 31 and are used to each house an O-ring 33 (FIG. 3C). When the nozzle 13 is inserted, four pressure gas inlet ports 34 (FIG. 2) of the nozzle 13 align with the major annular groove 31. The O-rings 33 (FIG. 3C) seal on either side of nozzle pressure ports 34 (FIG. 2) to prevent leakage of the pressurized gas. Pressure fittings 40, 41 are provided to supply high pressure for the nozzle 13 (not shown) and low pressure for counterflow gas. Inside the chamber 10, a shallow rectangular slot 37 may be provided to allow for positioning the counterflow tube 17 (FIG. 5C) opposing axially to the outlet of nozzle 13. An additional circular recess 38 may be provided to house an O-ring for a counterflow tube 17. A drain port 20 is located at the bottom of the chamber 10.

FIGS. 5A-5E provide cross-sectional views of the assembled chamber and cone assembly, provided with detail views demonstrating routing of pressurized gas within the chamber 10. FIG. 5A shows a front view of the chamber and cone assembly, including cross-sectional cut lines B-B and C-C, the cross-sectional views of which are respectively shown in FIGS. 5B and 5C. FIG. 5B shows a cross-sectional view of the chamber and cone assembly, taken along cut line B-B. FIG. 5C shows a cross-sectional view of the chamber and cone assembly, taken along cut line C-C.

FIG. 5D shows a detail view I from cross-section B-B of FIG. 5B, showing the high-pressure gas routing for the nozzle 13. The gas is delivered through pneumatic tubing connection, entering threaded nozzle port 40 to the major annulus 31 via a T-channel 42. In this way, high-pressure compressed gas is supplied via a threaded nozzle gas port 40 situated on the back of the chamber 10. The compressed gas quick connector for the nozzle 13 is connected to the threaded port 40, leading to a T-channel 42, that conducts the pressurized gas to the major annulus 31. The open end of the T-channel 42 is a machined access hole 44 to the major annulus 31 which is sealed permanently. Two minor annuli 32 are included on either side of major annulus 31, each of which may be configured to house an O-ring 33 (not shown).

FIG. 5E shows detail H from cross-section C-C of FIG. 5C, showing the base of counterflow tube 17. At the rear of chamber 10, a threaded counter flow gas port 41 is provided to supply low-pressure gas for counter flow tube 17 through a straight channel 43.

The T-channel 42 and straight channel 43 are designed for manufacturability. However, 3D printing technology may facilitate these features without the need to drill a channel to the annulus. These gas inlet ports 40, 41, and the nozzle insertion hole 30 (FIGS. 4B-C) are located such that nozzle pneumatic tubing 7 and counterflow pneumatic tubing 8, and the liquid capillary 6 are readily routed through the console 2 (FIG. 1). In addition, the location of these gas inlets 40, 41 at the lower rear of the aerosol chamber 10 minimizes the length of the pressurized gas tubing required while it facilitates sufficient articulation safety and ease of use of the aerosol generation and processing system 1.

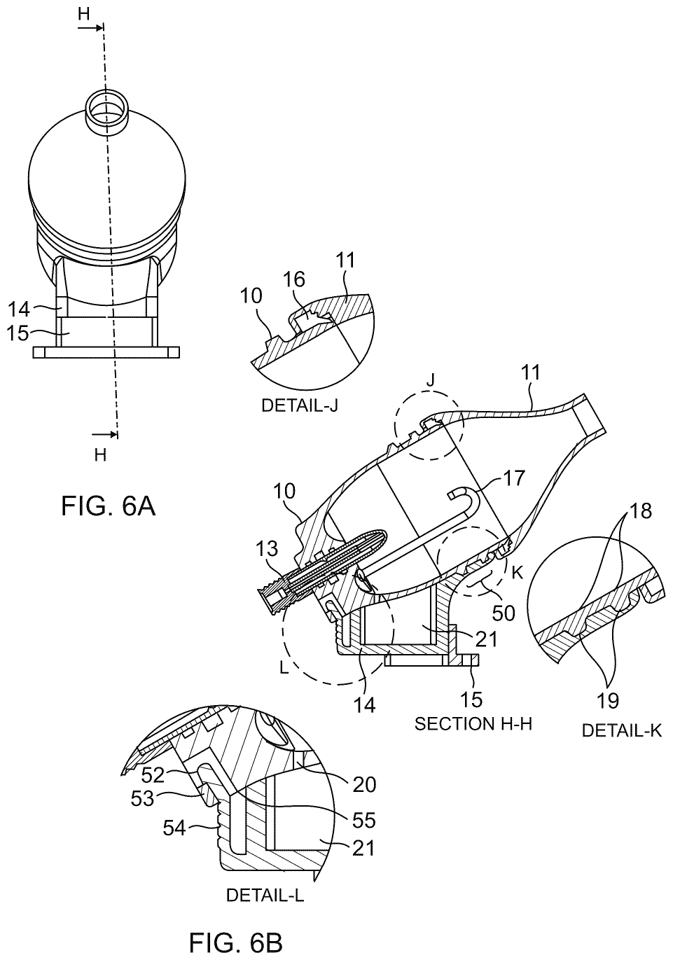

FIG. 6A shows a front view of the chamber and cone assembly on the pedestal 14 attached to the mounting bracket 15. A cross-sectional cut line H-H runs vertically through the chamber and cone assembly.

FIG. 6B shows sectional view H-H emphasizing the orientation and positioning of the nozzle 13 and the counterflow tube 17 within the chamber 10. The chamber 10 is shown atop pedestal 14 attached to the mounting bracket 15. The pedestal 14 may include a reservoir 21 to collect aerosol deposited within the chamber 11 and the cone 10, which drains through a drain port 20. If applicable, in addition to the deposited aerosol, also condensed liquid may be drained through the drain port 20. The aerosol chamber 10 and cone 11 combination when desired, is emplaced on a receptacle 50 that is readily attached to the mounting bracket 15 that is affixed atop the console 2 forming a pedestal 14. FIG. 6B includes three circled segments around the chamber 10, corresponding to detail view J, detail view K, and detail view L.

Detail J shows the connection point between the chamber 10 and the cone 11, in which a lip seal 16 enables attachment of the cone 11 with the chamber 10.

Detail K shows the grooves 19 on the pedestal 14 securely accepting the ribs 18 on the chamber 10. To emplace the aerosol generation and processing system 1 on the pedestal 14 on the console 2, a pair of concentric outer ribs 18 near the output of the aerosol chamber 10 are seated on corresponding crescent-shaped grooves 19 on the receptacle 50. This configuration stabilizes the positioning of the aerosol chamber 10 on the receptacle 50 at a 30° angle 22 and thus prevents unnecessary axial and lateral movement during use by the patient, wherein axial refers to the axis of the aerosol chamber 10 and cone 11. The 30° angle 22 can be modified based on the console height, pedestal design and application requirement of the aerosol therapy. Two ribs 18 were found to be sufficient to prevent lateral or axial movement of the chamber and cone assembly. According to alternative embodiments, other clamping mechanisms could be utilized, for instance mechanical or magnetic clamping mechanisms.

Detail L shows the connection between the chamber 10 and pedestal 14, in which a cantilevered clip 52 latches on a tab 53 in the back of the chamber 10. A knurled surface 54 on the cantilever clip 52 may be depressed to release the chamber 10 from the pedestal 14. A cantilevered clip 52 at the back of the pedestal 14 locks the chambers 10 in place when clipped on a tab 53 on the chamber 10. To disconnect the chamber from the mounting bracket 15 the user lifts and rotates the chamber up and gently backward about a fulcrum 55 while simultaneously depressing a textured grip 54 on the cantilevered clip 52 at the base of the pedestal 14. A small drain port 20 in the chamber allows drainage of aerosol deposited on the chamber walls into a reservoir 21 within the receptacle 50. The volume of the reservoir 21 in this invention is 12 ml, however, this volume is increased by enlarging or increasing the height of the pedestal 14. A fluid pump attached to the reservoir 21 prolongs the system operation by scavenging the deposited liquid in the reservoir 21. The drain port 20 is located at the lowest height inside the chamber 10 when positioned on the pedestal 14 at 30° inclination 22. The liquid flow through the drain port 20 may be modified by varying the port orifice diameter, or by modifying the surface tension of the port walls with a hydrophilic or hydrophobic coating. According to an alternative embodiment, a peristaltic pump may be connected to the drain port 20 through a flexible tube, which may be sufficient for draining the liquid from the chamber walls, thereby replacing the need for the reservoir 21 in the receptacle 50.

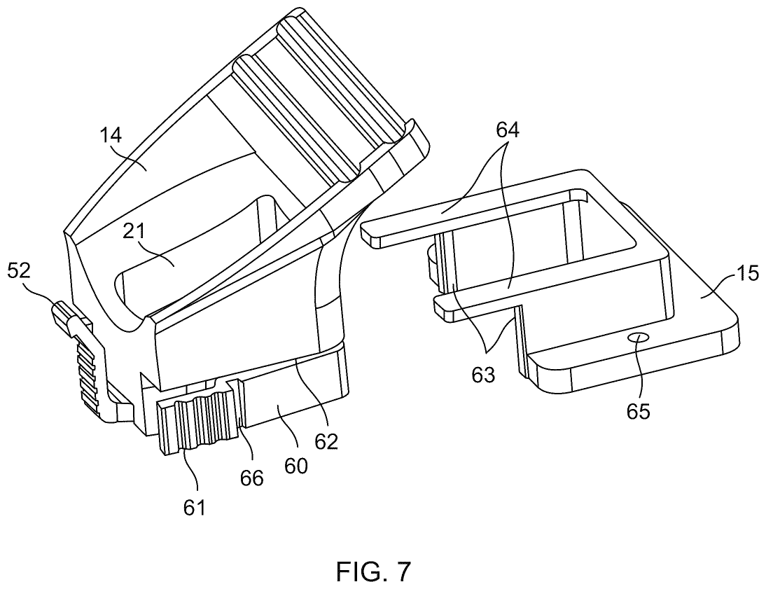

FIG. 7 shows an exploded view of the pedestal 14 and the mounting bracket 15 to which the pedestal 14 is configured to attach. To mount the aerosol generation and processing system 1 on the console 2, two cantilever clips 60 may be offset from base of pedestal 14. The cantilever clips 60 may be disposed on respective sides of the pedestal 14 and include textured grips 61, that are depressed for insertion into two rails 64 of the mounting bracket 15. These rails 64 ride on two horizontal slits 62 on the pedestal 14. In other words, the cantilevered clips 60 may form two slots 62 configured to accept corresponding rails 64 protruding horizontally from mounting bracket 15. Upon complete insertion, two vertical slots 66 on the sidewards cantilevered clips 60 of the pedestal 14 mate with corresponding vertical tabs 63 on the mounting bracket 15. The pedestal 14 is rigidly secured by sliding it on the mounting bracket 15 attached to the console using mounting bracket holes 65 or adhesive. A well may be formed in the body of the pedestal 14 in order to collect aerosol deposition draining from the chamber 10 (not shown) that is configured to be placed on the pedestal 14. At the rear of the pedestal 14, a cantilever vertical clip 52 may be included that clips on the back of chamber 10 to prevent its motion.

The pedestal 14, when needed, is removed from the mounting bracket 15 by depressing the horizontal cantilever clips 60 with two fingers, one for each cantilever. Two horizontal rails 64 on the mounting bracket 15 allow insertion and removal of the pedestal 14 and aerosol generation and processing system 1. These tab and rail combinations prevent movement of the pedestal 14 in all six degrees of freedom. Clearly, the configuration enables the aerosol generation and processing system 1 together with the adjoined pedestal 14 to be used flexibly by the patient. According to an alternative embodiment, not shown, magnetic latches may be implemented instead of cantilevered clips. For applications that require remote aerosol delivery to the patient or animal mouth or nose, the pedestal 14 can be modified as a hand-held attachment by increasing the lengths of the capillary 6 and gas tubing 7, 8. This enables free articulation and extension of the aerosol generation and processing system 1.

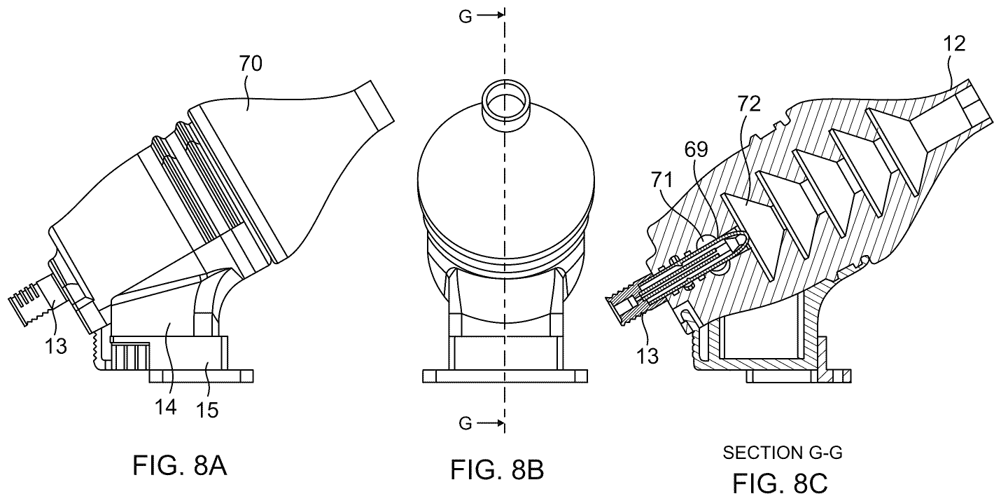

FIG. 8A shows a side view of another embodiment of the invention, including a compact multistage pod assembly having a compact multistage pod 70 placed on a pedestal 14 attached to a mounting bracket 15. A nozzle 13 is inserted at the rear of the compact multistage pod 70.

FIG. 8B shows a front view of the compact multistage pod assembly, shown with a vertical cross-sectional cut line G-G extending through the central axis.

FIG. 8C shows the sectional view G-G of FIG. 8B, which shows the reduced internal volume in the compact multistage pod 70. A series of conical cavities 72 are sequentially arranged in the compact multistage pod 70, corresponding to the space between the chamber and cone assembly in the conventional configuration (FIGS. 3A-C). The compact multistage pod 70 is an advantageous modification of the conventional chamber and cone assembly, particularly advantageous for use in cases where rapid on-and-off aerosol delivery is needed. This compact multistage pod 70 reduces the velocity of the aerosol plumes as they interact with each of up to five sequential of circumferential volumes with each internal periphery to reduce the velocity of the plume.

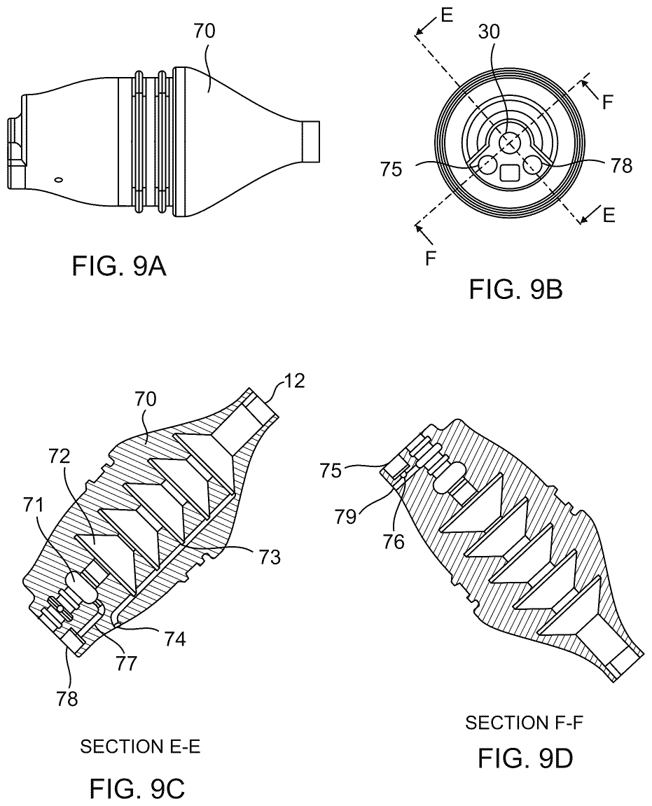

According to an alternative embodiment, a converging cross-sectional volume along the axis of the compact multistage pod 70 may be provided, which may be ideal for smaller tidal volumes for pediatric patients, as the effective internal volume of the compact-multistage pod 70 is reduced. The outer form of the compact multi-stage pod 70 may be externally identical to the conventional chamber-cone 10, 11, and thus may engage with the pedestal 14 in the same manner. According to an embodiment, the compact multistage pod 70 may be 3D printed, which enables fabrication of a rounded annulus cavity 71 for the dilution gas, the circumferential volumes, and a common drain channel 73 (FIG. 9C) without requiring conventional subtractive machining techniques. The configuration of the internal structure of the compact multistage pod 70 is further described with reference to FIG. 9A-9D.

FIG. 9A shows a side view of the compact multistage pod 70. FIG. 9B shows a rear view of the compact multistage pod 70 of FIG. 9A, including cross-sectional cut lines E-E and F-F, the cross-sectional views of which are respectively shown in FIGS. 9C and 9D. Collectively, FIGS. 9C-9D show the compact multistage pod 70 showing internal air channel routing. The rear of the compact multistage pod 70 includes a central cylindrical channel 30 configured to receive a nozzle 13 (not shown), a first threaded port 75 in connection with a T-channel 76 (FIG. 9D), and a second threaded port 78 in connection with an L-channel 77 (FIG. 9E) for routing dilution gas.

FIG. 9C shows the cross-sectional view of section E-E of FIG. 9B, demonstrating the internal structure for collecting aerosol deposition, while FIG. 9D shows the cross-sectional view of section F-F, showing the air channel routing for a nozzle 13 (not shown). A nozzle 13 may be inserted into a cylindrical hole 30 at the rear of the compact multistage pod 70. The compact multistage pod 70 maintains the same fixation mechanism to the pedestal 14 as in the conventional chamber and cone assembly. A common drain channel 73 collects liquid deposited at the walls from all conical cavities 72 to the drain port 74, and eventually to the reservoir 21 in pedestal 14 (FIG. 6B). The series of five circumferential volumes with internal conical-like cavities 72 are connected to a drain channel 73 that drains deposited aerosol liquid to the reservoir 21 within the pedestal 14 via the drain port 74. This embodiment of a compact multistage pod 70 eliminates the assembly of chamber, counterflow tube, lip seal and cone, resulting in a single piece construction with less parts. Furthermore, optimization of the internal volume of the compact multistage pod 70 is expected to decrease the velocity of the aerosol and so improve aerosol output characteristics. The rounded annulus cavity 71 near the nozzle tip collects the low-pressure gas (previously routed to the counter flow tube 17) and releases a sheath of gas through a thin cylindrical gap 69 between the nozzle barrel and the inside walls of the compact multistage pod 70. This gas sheath clears any potential deposition nearer the nozzle tip and assists as carrier gas for the aerosol downstream through conical cavities 72 of the multi-stage pod 70 and eventually through the mouthpiece 12. A T-channel 76 and an L-channel 77 provide pneumatic connections in the rounded annulus cavity 71 for high pressure nozzle ports and low-pressure carrier gas. The T-channel 76 and L-channel 77 are connected through adjacent pressure fittings attached to the threaded ports 75, 78, respectively. The T-channel 76 includes an open end 79, which may be permanently or temporarily capped after manufacturing.

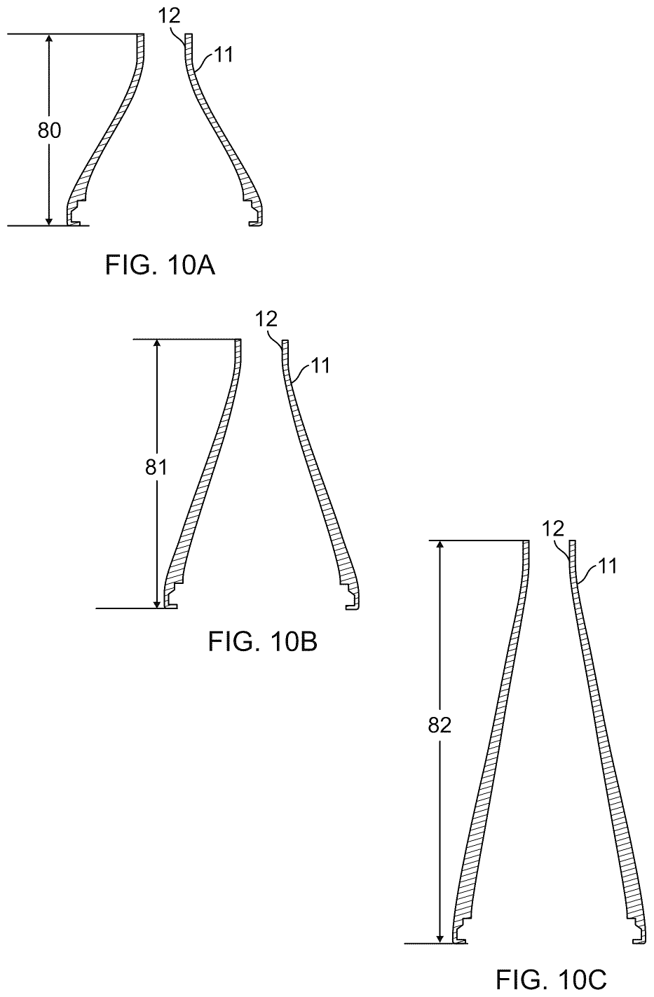

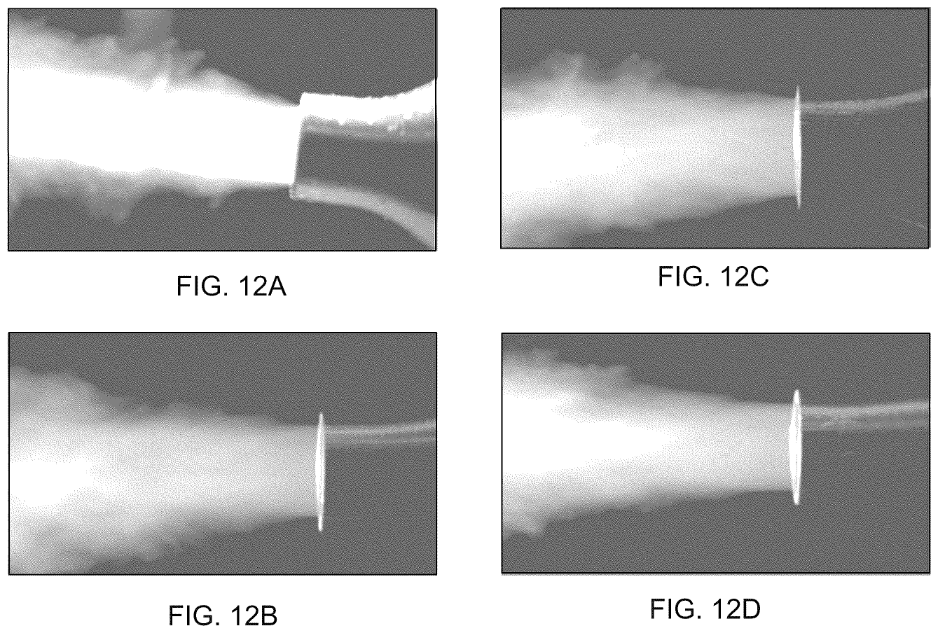

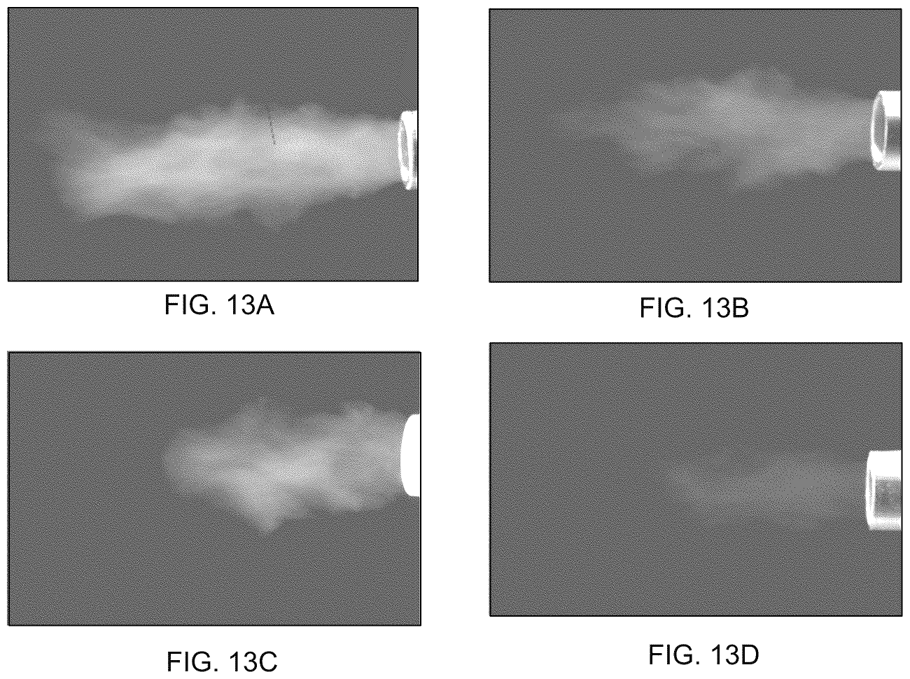

FIGS. 10A-10C respectively show cones 11 having varying lengths and correspondingly differing geometries, which affect the aerosol output characteristics. The effects of cone length and geometry are graphically represented in FIGS. 12 and 13. FIG. 10A shows a tapering cone 11 having a 70 mm length 80 converging to form a mouthpiece 12, the cone 11 configured to deliver aerosol. FIG. 10B shows a tapering cone 11 having a 100 mm length 81 converging to form a mouthpiece 12, the cone 11 configured to deliver aerosol. FIG. 10C shows a tapering cone 11 having a 150 mm length 82 converging to form a mouthpiece 12, the cone 11 configured to deliver aerosol.

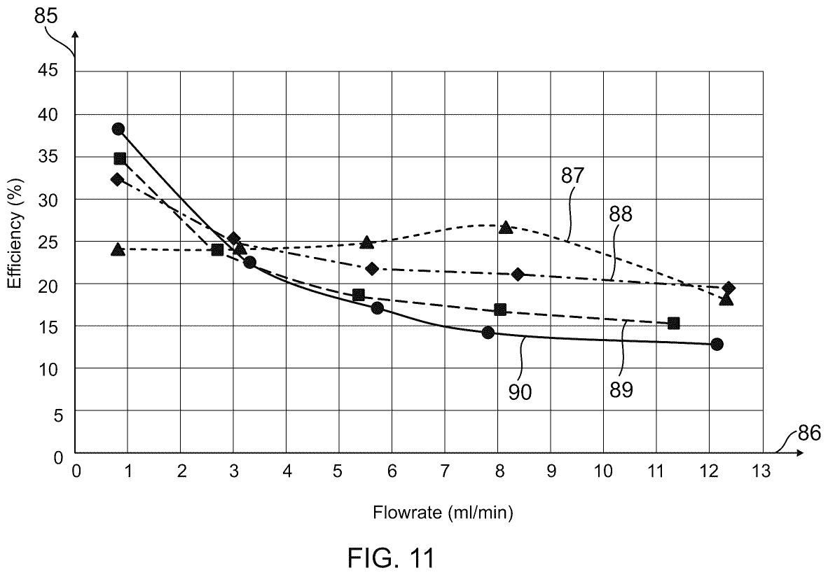

FIG. 11 shows a plot of flow rate 86 versus efficiency 85 for various delivery cone configurations, demonstrating the effect of flow rate 86 on output efficiency 85 for conventional cones 11 versus for compact multistage pods 70.

In this plot, the abscissa is the liquid flow rate 86 in units of ml/min, and the ordinate is the efficiency 85 given as a percent. The dotted line connecting the triangular data points 87 correspond to the compact multistage pod 70. The dot-dash line connecting the diamond data points 88 corresponds to a 70 mm length cone (FIG. 10A). The dashed line connecting the square data points 89 corresponds to a 100 mm length cone (FIG. 10B). The solid line connecting the circular data points 90 corresponds to a 150 mm length cone (FIG. 10C). It was found that at low liquid flow rates (1-3 ml/min) conventional chamber and cone design was more efficient (˜35%) compared to the compact multistage pod 70. At higher flow rates (>3 ml/min), the efficiency 85 was reduced for conventional chamber and cone design. However, moderate change in efficiency 85 was observed in the compact multistage pod 70. This efficiency 85 was calculated as:

$$η = \frac{{v}_{1} - {v}_{2}}{{v}_{1}}$$

Where ν₁ is the total volume of liquid aerosolized and ν₂ is the volume of deposited liquid collected from the chamber 10 after aerosolization. Latency between the aerosol onset command and achieving aerosol at the cone mouthpiece 12 was evaluated. The latency of aerosol output for on-demand aerosol delivery increases with increased cone length and internal volume. The latencies were 99, 118, 145 and 135 ms, for 70 mm, 100 mm, and 150 mm cones 12 (FIGS. 10A-C), and the compact multistage pod 70 respectively. It was observed that shorter 70 mm cone 12 (FIG. 10A) resulted in turbulent aerosol flow near the cone's outlet, compared to longer 150 mm cone 12 (FIG. 12D). As the length of the cones 12 increases, the turbulence of emanating aerosol decreases near the mouthpiece 12.

The plot shows that the efficiency 85 decreases as the flow rate 86 increases for the case of conventional chamber and cone 11 with counterflow tube 17, corresponding with data points 88, 89, 90. For the triangular data points 87 associated with the compact multistage pod 70, the efficiency 85 slightly increases and then decreases with flow rate 86.

FIGS. 12A-D show still images of aerosol expulsion, comparing a compact multistage pod 70 configuration against conventional cones 11 of various sizes, specifically showing aerosol output characteristics near the mouthpiece. FIG. 12A shows the compact multistage pod 70 and aerosol emanating from the mouthpiece. FIGS. 12B, 12C, and 12D presents aerosol emanating from 70 mm, 100 mm and 150 mm cones 11, respectively. The aerosol output is denser in FIG. 12A compared to FIGS. 12B, 12C, and 12D.

FIG. 13A shows a still image of aerosol emanating from the mouthpiece of compact multistage pod 70 when a 10 ms aerosol output command is provided to a microcontroller. FIGS. 13B, 13C, and 13D show still images of aerosol emanating from the mouthpiece of the conventional chamber and cone set-up with 70 mm, 100 mm, and 150 mm cone lengths (80, 81, 82 in FIGS. 10A-C), respectively, when a 10 ms aerosol output command is provided.

A test for observing aerosol characteristics at short bolus duration was conducted. During a 10 ms pulse, the aerosol bolus intensities were captured using a high-speed imaging camera. For all cases, the compact multistage pods 70, 70 mm, 100 mm, and 150 mm cones 11, the image frame with highest aerosol density was analyzed. The compact multistage pod 70 produced the highest density of aerosol flow near the cone outlet, whereas the 150 mm cone (FIG. 10C) produced the lowest density aerosol. This outcome indicates the compact multistage pod 70 design is useful for application where short bursts of aerosol are necessary, such as pediatric on-demand aerosol delivery. However, for the conventional chamber and cone configuration, the aerosol output characteristics and efficiency may be improved by optimizing internal shape of the chamber 10 and cone 11, and the length of the cone 11. The internal volume of multi-stage pod 70 design was 55 ml, whereas the internal volumes of the chamber-and-cone designs were 230, 270 and 330 ml for cone lengths of 70 mm (FIG. 10A), 100 mm (FIG. 10B) and 150 mm (FIG. 10C), respectively. The results of the tests captured in FIGS. 13A-D lead to the inference that larger internal volume of chamber 10 and cone 11 results in the dilution of aerosol boluses, resulting in lower aerosol intensity.

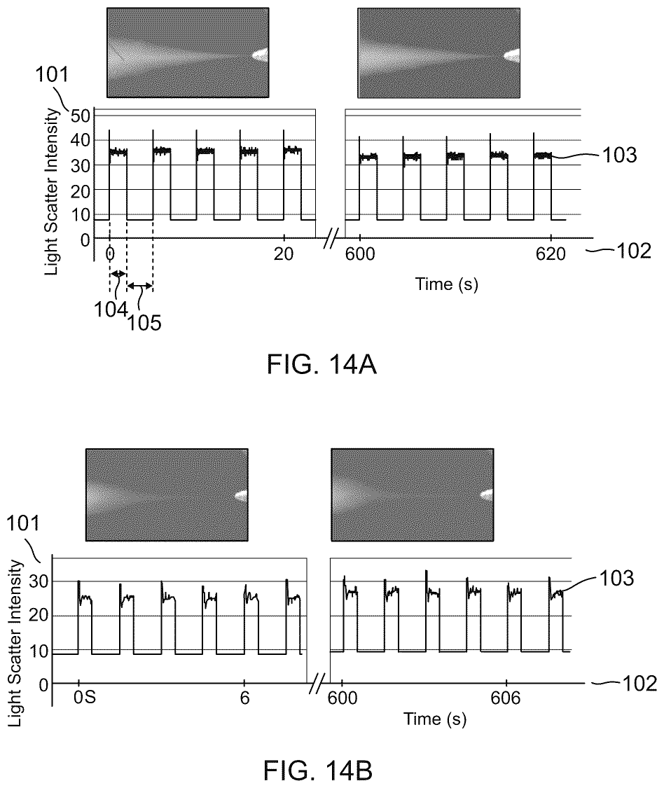

FIGS. 14A and 14B show the light scatter intensity 101 for each aerosol bolus period 103 given in arbitrary units on the ordinate, as a function of time 102 in units of seconds on the abscissa. The plotted data relates to aerosol boluses delivered through an AeroPulsR nozzle 13.

FIG. 14A shows data for a sequence in which aerosol generation is “on” for a duration of 2 seconds 104 and “off” for a duration of 3 seconds 105, with this sequence being repeated for a total duration of 10 minutes.

FIG. 14B shows data for a sequence in which aerosol generation is “on” for a duration of 0.5 seconds and “off” for a duration of 1 second, with this sequence being repeated for 10 minutes.

In the sequence plotted in FIG. 14A, the aerosol “on” duration 104 and “off” duration 105 was 2 seconds and 3 seconds, respectively. The result demonstrates that aerosols are precisely generated in boluses with similar intensity, even after 10 minutes of aerosolization. This degree of precision indicates a consistent dose delivery throughout the treatment period. Consistent delivery of boluses was also observed in the sequence plotted in FIG. 14B, where the aerosol “on” duration 104 and “off” duration 105 was 0.5 seconds and 1 second, respectively.

The flow rate of the aqueous 10% y-Globulins solution was maintained ˜3 ml/min. The nozzle 13 aerosolizes 100% of the solution. At 60 psi operating pressure, the aerosol concentration at the nozzle 13 was estimated to be 30 mg/l. Counterflow gas through the counterflow tube 17 was used to reduce the aerosol velocity and decreases wall deposition of aerosol emanating from the nozzle 13. This gas dilutes the aerosol concentration to 8 mg/l. With an estimated output efficiency of the system of 30%, the final concentration of the aerosol delivered is 2.4 mg/l. With reference to the sequence plotted in FIG. 14A, consider a case with a patient breathing 500 ml of tidal volume at a breath rate of 12 breaths/min. The total volume breathed is 6 liters. Using AcroPulsR, 14.4 mg/min of γ-Globulins is delivered. With an inline concentrator, e.g. a virtual impaction concentrator (not shown) located near the mouthpiece and having a cross-shaped slit, this concentration can be increased between 2 to 5 times, resulting in a concentration of 30 mg/ml to 70 mg/ml. The total dose of 1400 mg (i.e., 20 mg/kg for a 70 kg patient) is delivered within 20 minutes. Treatments requiring doses smaller than 1400 mg is accomplished in less than 20 minutes. This duration is reduced for patients with small tidal volumes such as infants and neonates. With reference to the sequence plotted in FIG. 14B, consider a smaller patient breathing 500 ml of air in one minute although with 30 breaths/min. With an output efficiency of 30%, the total dose delivered is calculated to be 3.3 mg/l. With a total volume of 0.5 1, 1.65 mg/min is delivered. Therefore, it would require less than 12 minutes to deliver a total dose of 20 mg/kg.

Although aerosol boluses of duration as small as 10 ms at 1 Hz were tested and observed using high speed imaging of the aerosol generation, consistent and repeatable aerosol boluses were obtained at a bolus duration of 250 ms aerosol at 1 Hz. These short duration boluses are expected to be consistent throughout a 10-20 minute treatment duration.

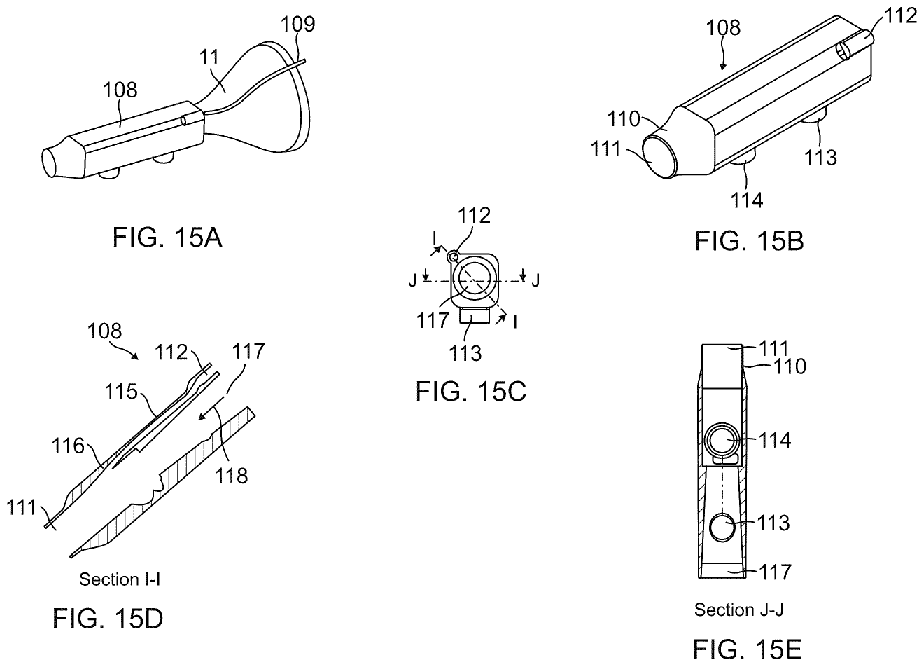

FIG. 15A shows a schematic of the modified bidirectional valve assembly 108 attached to the output cone 11. The modified bidirectional valve assembly 108 for automatic breath detection and aerosol delivery. A pneumatic tubing 109 extends from the modified bidirectional valve assembly 108 to the AcroPulsR console 2 (not shown) for connection with a pressure sensor.

FIG. 15B shows a perspective view of the modified bidirectional valve assembly 108. Aerosol is inhaled through the aerosol output end 111 via a mouthpiece 110. The pneumatic tubing 109 (shown in FIG. 15A) attaches to a connection port 112 on the modified bidirectional valve assembly 108. The modified bidirectional valve assembly 108 includes an inlet 117 for provision of aerosol, an outlet end 111 for the expulsion of aerosol, and a modified bidirectional valve mouthpiece 110 for inhalation. Inside the valve assembly there are two exhaust ports-first exhaust port 114 and second exhaust port 113, where one-way pressure relief valves with particulate filters are attached. These one-way pressure relief valves allow the flow of gases out of the exhaust ports 113, 114 while maintaining a safe operating pressure. First exhaust port 114 is for exhausting exhalation gases from the patients and is closest to the outlet 111 of the modified bidirectional valve assembly 108. The second exhaust port 113 is for exhausting gases, and is located closest to the inlet 117 (see FIGS. 15C-E) of the modified bidirectional valve assembly 108. A patient's exhalation gases 120 are released through a first exhaust port 114, while excess gas is released through second exhaust port 113 when the bidirectional valve flap 119 is in closed position.

FIG. 15C shows a rear view of the modified bidirectional valve assembly 108. The second exhaust port 113 is seen at the base of the modified bidirectional valve assembly 108. The modified bidirectional valve assembly inlet 117 is shown, including a diagonal cross-sectional line I-I and a horizontal cross-sectional line J-J. Connection port 112 is shown at the top left corner with diagonal cross-sectional line I-I passing through it.

FIG. 15D shows sectional view I-I of FIG. 15C of the modified bidirectional valve assembly 108. An internally routed conduit 115 connects a pressure sensor port 116 within the modified bidirectional valve 108 and a port 112 for the pressure sensor tubing. Aerosol 118 is configured to travel through the modified bidirectional valve assembly 108 from an input end 117 to an output end 111, the flow direction of which is depicted by an arrow.

FIG. 15E shows sectional view J-J of FIG. 15C, showing the internal top view of the modified bidirectional valve 108. The second exhaust port 113 is located proximate to the inlet end 117, while the first exhaust port 114 is located downstream of the second exhaust port 113, namely proximate to the outlet end 111 at which the modified bidirectional valve assembly mouthpiece 110 is located.

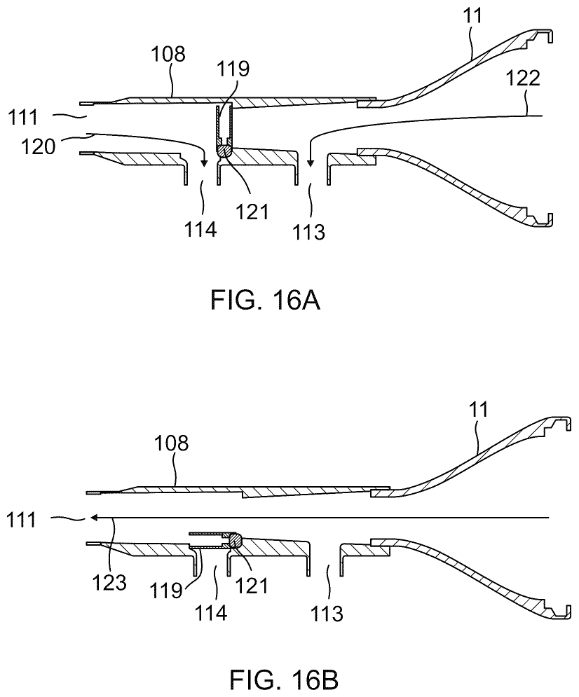

FIGS. 16A and 16B show cross-sectional side views of the modified bidirectional valve assembly 108 in a closed and open configuration, respectively. A pneumatically actuated flap 119 within the modified bidirectional valve assembly 108 may be configured to rotate 90° between two positions, namely a closed position shown in FIG. 16A and an open position shown in FIG. 16B.

FIG. 16A shows the flap 119 in closed position, blocking the gas flow through the modified bidirectional valve 108. The residual gases 122 generated from the AeroPulsR system are released through the second exhaust port 113, whereas the exhalation gases 120 and aerosol from the patient is released through a filter attached to the first exhaust port 114. A pneumatic actuator may be configured to rotate the flap 119 from its closed position to its open position about a pivot pin 121.

In the closed position of the flap 119, the aerosol and residual gases 122 emanating from the AeroPulsR system and flowing through the valve assembly 108 is blocked between the inlet 117 and outlet 111. The residual gases 122 from the AeroPulsR system are directed through the one-way pressure relief valve and a filter through the second exhaust port 113. Simultaneously, the exhalation gases 120 from the patient at the outlet end 111 are exhausted through the one-way pressure relief valve and a filter at the first exhaust port 114. Upon the initiation of inhalation by the patient, the pressure near the outlet 111 of the modified bidirectional valve assembly 108 decreases due to suction. This decrease in pressure is sensed by a pressure sensor routed through an inbuilt internal conduit 115 (shown in FIG. 15) within the modified bidirectional valve assembly 108. A microcontroller unit subsequently may activate the controllable valve to pneumatically rotate the flap 119 by 90° into its open position.

FIG. 16B shows the open position of the flap 119, which enables no-resistance flow of aerosol 123 generated by AeroPulsR through the modified bidirectional valve 108. At this open position of the flap 119, the first exhaust port 114 is closed and aerosolization is activated. Constant aerosol delivery is maintained by the AcroPulsR system until the completion of the inhalation. Post-inhalation, the pressure within the modified bidirectional valve 108 increases, this increased pressure is sensed by the pressure sensor, which enables rotation of the controllable valve 90° to its closed position. At this position, the aerosol generation and delivery are stopped by AeroPulsR, And the first exhaust port 114 is opened, enabling free flow of patient's exhalation gases 120 through the one-way pressure relief valve and the filter.

The flap 119 may be configured to be pneumatically rotated to an open position simultaneously with aerosol generation and delivery when inhalation is detected, and pneumatically rotate to a closed position such that aerosol generation and delivery is stopped. According to an embodiment, the flap 119 may be configured to open and close with a time interval offset from aerosol generation and termination, respectively. This offset may account for time taken for the aerosol to travel from the nozzle outlet to the modified bidirectional valve assembly 108, for instance due to intermediate dead spacing of the delivery tubing.

### LIST OF REFERENCE NUMERALS

1 aerosol generation and processing system
2 console
3 dispenser system
4 device control panel
5 parameter input panel
6 liquid capillary
7 pneumatic tubing for nozzle
8 pneumatic tubing for counterflow
9 one-way check valve
10 chamber
11 cone
12 mouthpiece
13 nozzle
14 pedestal
15 mounting bracket
16 lip seal
17 counterflow tube
18 concentric outer ribs on chamber
19 crescent shaped grooves on pedestal
20 chamber drain port
21 reservoir
22 30° angle
30 cylindrical channel
31 major annular groove
32 minor annular groove
33 O-ring
34 nozzle pressure port
40 threaded nozzle gas port
41 threaded counterflow gas port
42 T-channel
43 straight channel
44 machining access hole
50 receptacle
52 rear cantilevered clip on pedestal
53 rear tab on chamber
54 textured grip on the cantilevered grip
55 rear fulcrum on chamber
60 sideward cantilever clip on pedestal
61 textured grip on the sideward cantilever clip on pedestal
62 horizontal slits on the pedestal
63 vertical tab on the mounting bracket
64 rails on the mounting bracket
65 mounting bracket holes
66 vertical slot on the sideward cantilevered clip on pedestal
69 thin cylindrical gap
70 compact multistage pod
71 rounded annulus cavity
72 conical cavities
73 drain channel in compact multistage pod
74 drain hole in compact multistage pod
75 threaded port for nozzle gas
76 T-channel for nozzle gas in the compact multistage pod
77 L-channel
78 threaded port for dilution gas
79 open end of T-channel in compact multistage pod
80 70 mm cone
81 100 mm cone
82 150 mm cone
85 efficiency
86 liquid flow rate
87 data points for compact multistage pod
88 data points for 70 mm cone
89 data points for 100 mm cone
90 data points for 150 mm cone
101 light scatter intensity
102 time
103 aerosol bolus period
104 aerosol “on” duration
105 aerosol “off” duration
106 vial
108 modified bidirectional valve assembly
109 pneumatic tubing
110 modified bidirectional valve assembly mouthpiece
111 outlet of modified bidirectional valve assembly
112 port for pneumatic tubing
113 second exhaust port
114 first exhaust port
115 internal conduit
116 pressure sensor port
117 inlet of the modified bidirectional valve assembly
118 aerosol
119 flap
120 exhalation gases
121 pivot pin
122 residual gases
123 aerosol gas flow from aerosol generation and processing system

What is claimed is:

1. An aqueous aerosol generation and delivery system for high dose aerosol delivery, said system comprising:
a multistage pod having an inner cavity comprising in series a plurality of sequentially arranged multi-stage conical cavities having the shape of truncated cones narrowing in a direction of flow of the aerosol, said multistage pod further comprising:
an input end comprising an aerosolizing nozzle having a fluid input port and being inserted into a cylindrical channel and, a nozzle gas port, and a dilution gas port configured to receive an aerosolizing nozzle; and
an output end comprising an output opening through which aerosol is delivered;
wherein the aerosolizing nozzle is configured to generate and expel aerosol through a nozzle tip;
a rounded annulus cavity disposed within the multistage pod proximate to the nozzle tip, said rounded annulus cavity configured to direct dilution gas towards the nozzle tip; and
said plurality of sequentially arranged multi-stage conical cavities are disposed between the nozzle tip and the output end within the multistage pod, wherein the multi-stage conical cavities are arranged coaxial with the nozzle, and wherein the multi-stage conical cavities are configured to decrease the velocity of the aerosol plume by having said shape of truncated cones narrowing in a direction of flow of the aerosol, such that the system expels a dense column of soft-flowing aerosol suitable for inhalation by a patient;
wherein the input end of the multistage pod further includes:
a nozzle gas port configured to supply high-pressure gas to a nozzle annulus via a T-channel, said nozzle annulus circumferentially surrounding the plurality of nozzle gas input holes; and
a dilution gas port configured to supply low-pressure gas to a rounded annulus cavity via an L-channel.

2. The aqueous aerosol generation and delivery system according to claim 1, further comprising:
a single internal drain channel connecting the conical cavities, said drain channel configured to collect and direct aerosol deposition out of a unitary housing via a drain hole formed in the unitary housing.

3. The aqueous aerosol generation and delivery system according to claim 1, further comprising a modified bidirectional valve assembly for ventilation of exhalation, said modified bidirectional valve assembly comprising:
a valve inlet end and a valve outlet end, wherein the valve inlet end is attached to the output end of the unitary housing;
a first exhaust port near the valve inlet end, said first exhaust port configured for releasing residual gases from the aqueous aerosol generation and delivery system;
a second exhaust port near the valve outlet end, said second exhaust port configured for releasing patient exhalation gases; and
a pneumatically-operated flap configured to rotate between a closed position and an open position,
wherein in the closed position, the second exhaust port is open for resistance-free flow of patient exhalation gases, and a direct path between the valve inlet end and the valve outlet end is completely blocked; and
wherein in the open position, the second exhaust port is closed, and the direct path between the valve inlet end and the valve outlet end is open for aerosol delivery.

4. The aqueous aerosol generation and delivery system according to claim 3, wherein the modified bidirectional valve assembly further comprises an internal conduit having a conduit inlet near the valve outlet end and a conduit outlet near the valve inlet end, wherein the conduit outlet is connected to a pressure sensor; and
wherein the system is configured to:
monitor the pressure near the valve outlet end;
trigger aerosol generation when the pressure near the valve outlet end decreases due to inhalation, and
stop aerosol generation when the pressure near the valve outlet end increases due to exhalation.

5. The aqueous aerosol generation and delivery system according to claim 1, further comprising:
a pedestal configured to support a unitary housing at an angle, said pedestal comprising:
at least one groove configured to interface with at least one external rib on the unitary housing;
an internal reservoir configured to collect aerosol deposition from an inner wall of the unitary housing.

6. The aqueous aerosol generation and delivery system according to claim 5, wherein the pedestal is secured to a console using a mounting bracket.

7. The aqueous aerosol generation and delivery system according to claim 6, wherein the pedestal further comprises:
a top surface contoured to receive the chamber;
a cantilevered vertical clip at the rear of the pedestal, configured to latch onto a tab on the unitary housing;
at least two grooves configured to interface with corresponding ribs on the unitary housing;
at least two slits, one on each lateral side of the pedestal, configured to allow the pedestal to slide onto the mounting bracket rigidly attached at the top of the console; and
at least two cantilevered horizontal clips including integrated slots configured to latch onto the mounting bracket when the horizontal clips are depressed for insertion onto the mounting bracket.

8. The aqueous aerosol generation and delivery system according to claim 5, wherein the unitary housing further comprises a drain port for draining aerosol deposition from the unitary housing into the inner reservoir of the pedestal, wherein the drain port includes a hydrophilic coating.

9. The aqueous aerosol generation and delivery system according to claim 5, wherein the mounting bracket comprises:
a plurality of through holes for rigidly attaching the mounting bracket to the console;
at least two horizontal rails configured to slide along the slits on the pedestal; and
at least two vertical tabs configured to seat into the corresponding slots on the cantilevered horizontal clips of the pedestal.

10. The aqueous aerosol generation and delivery system of claim 1, further comprising:
a capillary configured to convey a to-be-aerosolized liquid from a vial to the nozzle input port;
a first pneumatic tube configured to supply high-pressure gas to the nozzle gas port; and
a second pneumatic tube configured to supply low-pressure gas to the dilution gas port.

11. An aqueous aerosol generation and delivery system comprising:
a chamber having a chamber input end and a chamber output end;
an aerosol generating nozzle configured to be inserted into a nozzle input port formed in the chamber input end, said nozzle comprising a nozzle barrel having a plurality of circumferentially arranged nozzle gas input holes;
a cone having a first cone end and a second cone end, wherein:
the first cone end is configured to attached to the chamber output end;
the diameter of the first cone end is greater than the diameter of the second cone end; and
the second cone end includes an opening configured to expel aerosol generated by the nozzle; and
a counterflow tube arranged within the chamber, said counterflow tube configured to eject pressured gas coaxially in the opposite direction of the nozzle to decelerate the high velocity aerosol emanating from the nozzle;
wherein the chamber input end further includes:
a nozzle gas port configured to supply high-pressure gas to a nozzle annulus via a T-channel, said nozzle annulus circumferentially surrounding the plurality of nozzle gas input holes; and
a counterflow gas port configured to supply low-pressure gas to the counterflow tube via a straight channel; wherein
the nozzle gas port and the counterflow gas port are separate ports that are not interconnected;
the counterflow gas port is the only source of dilution gas; and
the system is configured to expel a dense column of soft-flowing aerosol suitable for inhalation by a patient.

12. The aqueous aerosol generation and delivery system of claim 11, further comprising:
a capillary configured to convey a to-be-aerosolized liquid from a vial to the nozzle input port;
a first pneumatic tube configured to supply high-pressure gas to the nozzle gas port; and
a second pneumatic tube configured to supply low-pressure gas to the counterflow gas port.

13. The aqueous aerosol generation and delivery system of claim 11, wherein the counterflow tube is J-shaped, and comprises a base portion extending parallel to the nozzle, and a curved portion terminating coaxially to the nozzle.

14. The aqueous aerosol generation and delivery system according to claim 11, wherein the cone is attached to the chamber output end through a lip seal.

15. The aqueous aerosol generation and delivery system of claim 11, wherein a mouthpiece is provided at the second cone end.

16. The aqueous aerosol generation and delivery system according to claim 11, further comprising a pedestal configured to support the chamber at an angle, said pedestal comprising:
at least one groove configured to interface with at least one external rib on the chamber;
an internal reservoir configured to collect aerosol deposition from an inner wall of the chamber.

17. The aqueous aerosol generation and delivery system according to claim 16, wherein the pedestal is secured to a console using a mounting bracket.

18. The aqueous aerosol generation and delivery system according to claim 17, wherein the pedestal further comprises:
a top surface contoured to receive the chamber;
a cantilevered vertical clip at the rear of the pedestal, configured to latch onto a tab on the chamber;
at least two grooves configured to interface with corresponding ribs on the chamber;
at least two slits, one on each lateral side of the pedestal, configured to allow the pedestal to slide onto the mounting bracket rigidly attached at the top of the console; and
at least two cantilevered horizontal clips including integrated slots configured to latch onto the mounting bracket when the horizontal clips are depressed for insertion onto the mounting bracket.

19. The aqueous aerosol generation and delivery system according to claim 16, wherein the chamber further comprises a drain port for draining aerosol deposition from the chamber into the inner reservoir of the pedestal, wherein the drain port includes a hydrophilic coating.

20. The aqueous aerosol generation and delivery system according to claim 17, wherein the mounting bracket comprises:
a plurality of through holes for rigidly attaching the mounting bracket to the console;
at least two horizontal rails configured to slide along the slits on the pedestal; and
at least two vertical tabs configured to seat into the corresponding slots on the cantilevered horizontal clips of the pedestal.

21. The aqueous aerosol generation and delivery system according to claim 17, wherein the console is configured to house aerosol generating pneumatic hardware, a pneumatic control system, and a microcontroller circuit for controlling the pneumatic hardware; and the console further comprises:
a parameter input panel configured for aerosol therapy parameter input; and
a device control panel comprising a main power button, a therapy initiation button, and a plurality of multi-colored pilot lights configured to guide configured to indicate inhalation duration and exhalation duration.

22. The aqueous aerosol generation and delivery system according to claim 11, further comprising a modified bidirectional valve assembly for ventilation of exhalation, said modified bidirectional valve assembly comprising:
a valve inlet end and a valve outlet end, wherein the valve inlet end is attached to the second cone end;
a first exhaust port near the valve inlet end, said first exhaust port configured for releasing residual gases from the aqueous aerosol generation and delivery system;
a second exhaust port near the valve outlet end, said second exhaust port configured for releasing patient exhalation gases;
a pneumatically-operated flap configured to rotate between a closed position and an open position,
wherein in the closed position, the second exhaust port is open for resistance-free flow of patient exhalation gases, and a direct path between the valve inlet end and the valve outlet end is completely blocked; and
wherein in the open position, the second exhaust port is closed, and the direct path between the valve inlet end and the valve outlet end is open for aerosol delivery.

23. The aqueous aerosol generation and delivery system according to claim 22, wherein the modified bidirectional valve assembly further comprises an internal conduit having a conduit inlet near the valve outlet end and a conduit outlet near the valve inlet end, wherein the conduit outlet is connected to a pressure sensor; and
wherein the system is configured to:
monitor the pressure near the valve outlet end;
trigger aerosol generation when the pressure near the valve outlet end decreases due to inhalation; and
stop aerosol generation when the pressure near the valve outlet end increases due to exhalation.
